(19) United States
(12) Patent Application Publication
(10) Pub. No.: US 2017/0080142 A1
(43) Pub. Date: Mar. 23, 2017

(54) FILTRATION OF CIRCULATING TUMOR CELLS FOR THERAPUTIC PURPOSES

(71) Applicant: Oded Biran, Lawrence, NJ (US)

(72) Inventor: Oded Biran, Lawrence, NJ (US)

(21) Appl. No.: 14/862,898

(22) Filed: Sep. 23, 2015

(51) Int. Cl. A61M 1/36; B03C 1/005; A61M 1/34

(52) U.S. Cl. CPC A61M 1/3618; A61M 1/34; A61M 1/3621; B03C 1/005

(57) ABSTRACT

The present invention provides a method for filtration of CTCs directly out of patient blood and returning the blood after filtration to the patient. CTCs are an important factor for diagnosis and prognosis of cancer patients and can cause cancer metastasis. By eliminating CTCs from the bloodstream the chances for metastasis reduction decrease.

### FIELD OF THE INVENTION

The present invention relates to methods for mechanically filtering tumor cells that were shedded into the blood stream from a malignant tumor. Specifically the invention relates to utilizing and enhancing technologies for therapeutic—filtration use.

### BACKGROUND OF THE INVENTION

Circulating Tumor Cells (CTCs) are cells shedded by malignant tumors into the human bloodstream and at times, the lymphatic system. They have been identified as key diagnostic and prognostic indicators, as well as the origins of new metastatic sites in metastatic cancers.

Various methods have been utilized to “filter” CTCs out of blood samples for diagnostic and prognostic purposes, some are based on the CTCs physical traits (mass, size, shape)—these are known as microfluidic methods, some are based on CTCs connecting to biological receptors—these are known as the enrichment methods, and some are based on connection of magnetic beads to CTCs and then filtering out CTCs with the use of magnets—known as immunomagnetic methods. All of these methods rely on utilization of an amount of blood being drawn from a patient and extraction of CTCs from that blood.

While a variety of cancerous tumors shed CTC, the cancers most identified with CTCs are metastatic breast cancer, with approximated 234,000 new cases in 2015, colorectal cancer, with approximated 140,000 new cases in 2015, and prostate cancer, with approximated 220,000 new cases in 2015.

The most commonly used method for separating CTCs from blood is the CellSearch method developed by Veridex, In the CellSearch® system, after an automated separation of cells from plasma obtained from 7.5 ml of blood, CTCs are magnetically captured trough a ferrofluid-coupled antibody against EpCAM, and then sequentially permeabilized, fixed, and labeled with the fluorescent nuclear dye DAPI and fluorescent antibodies to the leukocyte marker CD45 and to epithelial markers cytokeratins (CK) 8, 18, and 19. The treated sample is then loaded into a cartridge where strong magnetic force attracts the immunomagnetically-labeled cells for analysis by the Cell-Tracks AnalyzerVR, a semi-automated fluorescence microscope that scans the sample at four different wavelengths, records the fluorescent events, and automatically presents images in a gallery format for classification by trained operators.

Accumulated efforts have been invested in finding a technology to separate CTCs from blood more efficiently and quickly, however, to my knowledge, to date, no one has looked as removal of CTCs from the bloodstream as a therapeutic process.

### SUMMARY OF THE INVENTION

A novel method of reducing the spread of metastatic cancers by filtering CTCs from cancer patients' blood and returning the blood to the patient, filtered, utilizing microfluidic chambers and/or filters and/or acoustic waves and/or enrichment techniques, .

Thus, in a first aspect, the present invention provides a method for classifying a thyroid lesion sample, the method comprising the steps of:
a. Drawing a patients' blood and channeling it, either pressurized or not-pressurized, through a machine which will contain one or more microfluidic filters and/or utilize acoustic waves and/or will have one or more surfaces covered in antibodies relevant for CTC-extraction enrichment techniques;
b. returning the filtered blood to the patient;

### DETAILED DESCRIPTION OF THE INVENTION

Despite accumulated efforts in the search for methods to diminish tumors metastasizing, a method for doing so has not yet been contemplated, until now. The method for CTC removal includes a machine that in certain embodiments, is made of a combination of previously known technologies for filtering CTCs out of blood.

In certain embodiments, the machine is made of a single, previously known technology for filtering CTCs.

In certain embodiments, the machine is made of a single, previously known technology for filtering CTCs, which is repeated over and over again to create a flow of blood volume to provide for timely filtration of a patients complete blood supply.

In certain embodiments, the machine is made of a single, previously known technology for filtering CTCs, which is duplicated or multiplied to create a flow of blood volume to provide for timely filtration of a patients complete blood supply.

In certain embodiments, the machine is made of multiple identical or non-identical microfluidic chambers, tubes and/or filters coated or not coated with complementary receptors and is either enhanced or not enhanced by the utilization of acoustic waves.

In certain embodiments, the machine is made of multiple identical or non-identical microfluidic chambers, tubes and/or filters coated or not-coated with CTC complementary receptors and is either enhanced or not enhanced by the utilization of acoustic waves, which tubes or chambers are either magnetized or not magnetized.

In certain embodiments of the invention, the blood is treated in a specific manner prior to its entrance into the machine.

In certain embodiments of the invention, magnetic beads are attached via complementary receptors on the CTCs prior to the bloods' entrance into the machine.

In certain embodiments of the invention, the blood undergoes a centrifugal process prior to its entrance into the machine.

In certain embodiments of the invention, the blood is separated to various components prior its' entrance into the machine.

It is to be understood that the terminology used herein is for the purpose of describing particular embodiments only and it is not intended to be limiting. It must be noted that, as used in the specification and the appended claims, the singular forms “a,” “an” and “the” include plural referents unless the context clearly dictates otherwise.

As used herein, the term “about” refers to +/−10%.

“Attached” or “immobilized”, as used herein to refer to a probe and a solid support, means that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

“Biological sample” or “sample”, as used herein, means a sample of biological tissue or fluid that comprises nucleic acids, and/or cells. Such samples include, but are not limited to, tissue or fluid isolated from subjects.

“Complement” or “complementary”, as used herein to refer to a nucleic acid, may mean Watson-Crick (e.g., A-T/U and C-G) or Hoogsteen base pairing between nucleotides or nucleotide analogs of nucleic acid molecules. A full complement or fully complementary means 100% complementary base pairing between nucleotides or nucleotide analogs of nucleic acid molecules. In some embodiments, the complementary sequence has a reverse orientation (5′-3′).

As used herein, the term “stage of cancer” refers to a numerical measurement of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “subject” refers to a mammal, including both human and other mammals. The methods of the present invention are preferably applied to human subjects.

As used herein, the term “Microfluidics” refers to systems, devices, and methods for processing small volumes of fluids. Because microfluidic systems can integrate a wide variety of operations to manipulating fluids, such as chemical or biological samples, these systems have many application areas, such as biological assays (for, e.g., medical diagnoses and drug delivery), biochemical sensors, or life science research in general.

As used herein, the term “subtype of cancer” refers to different types of cancer that affect the same organ (e.g., papillary, follicular carcinoma and follicular variant papillary carcinoma of the thyroid).

In another aspect, the present invention provides a method for recovering large amounts of CTCs for diagnostic purposes in a timely manner

In another further aspect, the present invention provides a method for recovering large amounts of CTCs for prognostic purposes in a timely manner.

1. A method for filtering blood through a machine that filters CTCs for the purpose of reducing cancer metastasis, that method containing the following steps:
a. Extracting a patients' blood through tubing;
b. Funneling the blood through a machine that filters out CTCs;
c. Returning the blood, without the CTCs, to the patient;

2. A method for filtering blood through a machine that filters CTCs for the purpose of diagnosis, that method containing the following steps:
a. Extracting a patients' blood through tubing;
b. Funneling the blood through a machine that filters out CTCs;
c. Returning the blood, without the CTCs, to the patient;

3. A method for filtering blood through a machine that filters CTCs for the purpose of prognosis, that method containing the following steps:
a. Extracting a patients' blood through tubing;
b. Funneling the blood through a machine that filters out CTCs;
c. Returning the blood, without the CTCs, to the patient;

4. The method of claims 1-3, wherein following step (a) or (b) the patients' blood undergoes an additional process comprising of enrichment with magnetic microbeads in a separate machine or chamber.

5. The method of claims 1-4, wherein following step (a) or (b) the patients' blood undergoes an additional process comprising of separation of the blood into its separate components in a separate machine or chamber.

6. The method of claims 1-4, wherein prior to its entrance into the machine in step (b), the patients' blood undergoes an additional process comprising of separation of the blood into its separate components in a separate machine or chamber.

7. The method of claims 1-6, wherein the machine in step (b) is made of a single, previously known technology for filtering CTCs, which is duplicated or multiplied to create a flow of blood volume to provide for timely filtration of a patients complete blood supply.

8. The method of claims 1-7, wherein the machine in step (b) is made of multiple identical microfluidic chambers, tubes or filters.

9. The method of claims 1-7, wherein the machine in step (b) is made of multiple non-identical microfluidic chambers, tubes and/or filters.

10. The method of claims 1-9, wherein surfaces of the machine in step (b) are covered with CTC complementary receptors.

11. The method of claims 1-10, wherein the machine in step (b) utilizes acoustic waves to enhance its effectiveness.

12. The method of claims 1-11, wherein the machine in step (b) utilizes magnetic fields to enhance its effectiveness.

13. The method of claims 1-12, wherein the machine in step (b) utilizes magnetized surfaces to enhance its effectiveness.

14. The method of claims 1-12, wherein the blood flow through the machine is pressurized.
